(19) United States
(12) Patent Application Publication
(10) Pub. No.: US 2016/0374576 A1
(43) Pub. Date: Dec. 29, 2016

(54) LOW DRIFT IMPLANTABLE PRESSURE SENSORS

(71) Applicant: Purdue Research Foundation, West Lafayette, IN (US)

(72) Inventors: Babak Ziaie, West Lafayette, IN (US); Albert Kim, West Lafayette, IN (US)

(73) Assignee: Purdue Research Foundation, West Lafayette, IN (US)

(21) Appl. No.: 15/191,880

(22) Filed: Jun. 24, 2016

Related U.S. Application Data
(60) Provisional application No. 62/184,214, filed on Jun. 24, 2015.

(51) Int. Cl. A61B 5/03; A61B 5/00

(52) U.S. Cl. CPC A61B 5/036; A61B 5/6853; A61B 5/6885; A61B 2562/168; A61B 2562/0247; A61B 2562/187; A61B 2562/222

(57) ABSTRACT

An implantable pressure sensor arrangement is disclosed. The sensor arrangement includes a pressure sensor, a cable having a plurality of conductors coupled to the pressure sensor, a flexible balloon partially fitted around the subassembly, thereby encasing the pressure sensor while each end of the cable is extending away from the balloon, the flexible balloon is then filled with an incompressible fluid, thereby allowing pressure changes outside of the balloon to be sensed by the pressure sensor inside of the balloon.

### CROSS-REFERENCE TO RELATED APPLICATIONS

The present U.S. patent application is related to and claims the priority benefit of U.S. Provisional Patent Application Ser. No. 62/184,214, filed Jun. 24, 2015, the contents of which are hereby incorporated by reference in their entirety into the present disclosure.

### TECHNICAL FIELD

The present disclosure generally relates to pressure sensors, and in particular to pressure sensors with low drift.

### BACKGROUND

This section introduces aspects that may help facilitate a better understanding of the disclosure. Accordingly, these statements are to be read in this light and are not to be understood as admissions about what is or is not prior art.

Monitoring pressures and pressure changes in a human body is typically of interest in various medical applications. In some cases, one or more pressure sensors may be implanted in a or on an organ to sense pressure and/or pressure change within the organ over an extended time period.

Pressure in various organs, such as brain, heart, eyes, bladder, gastrointestinal tract, etc. carries important indications of health. Specially, some of those pressures are needed to be monitored continuously over a long period of time (months to years). Researchers have developed implantable pressure monitoring system based on piezoresistive or capacitive transduction methods to meet the above-mentioned needs. However, one remaining challenge is to develop a low drift implantable pressure monitoring systems for long-term applications. The main source of drift in implantable pressure sensors is absorption of water and other tissue fluids (ambient factors) into the polymeric material used in packaging the sensor. This causes swelling, creep, or corrosion which overtime puts the sensing membrane under unknown stresses causing the drift. Base line drift is in particular difficult to compensate for since it can occur in either direction (positive and negative) and is not predictable. Once the sensor drifts, it requires re-calibration, which in many clinical applications is not easy to accomplish (e.g., pressure sensors implanted in brain ventricles to monitor intracranial pressure). One solution to this challenge, adopted by, e.g., DATA SCIENCES (St. Paul, Minn.), is to house the sensor in a hard shell (ceramic or metallic) package (i.e., physically isolate it from body fluids in the main electronic module) and connect it to the site of measurement through a gel filled catheter. This approach utilizes a technique that includes filling small catheters with gel with limitations as to adaptability for larger animals and humans. In addition, the gel-filled catheter needs to stay patent (un-clogged) throughout the device's lifetime, something that can be challenging in many physiological environments. Other methods investigated rely on mechanical design to isolate the sensing membrane from external forces, use of multiple sensors in a package, and application of an electrostatic force to the membrane in order to re-calibrate the sensor. So far, due to complexity and cost none of these methods have solved the drift challenge.

Therefore, there is an unmet need for an implantable pressure sensor that addresses the drift challenge in existing implantable pressure sensors.

### SUMMARY

An implantable pressure sensor arrangement is disclosed. The sensor arrangement includes a pressure sensor, a cable having a plurality of conductors coupled to the pressure sensor, forming a sensor-cable subassembly with the cable positioned about the sensor providing two ends, a flexible balloon partially fitted around the subassembly, thereby encasing the pressure sensor while each end of the cable is extending away from the balloon, the flexible balloon is then filled with an incompressible fluid, thereby allowing pressure changes outside of the balloon to be sensed by the pressure sensor inside of the balloon.

### BRIEF DESCRIPTION OF DRAWINGS

FIG. 1 is a schematic view of a disclosed sensor arrangement including: a pressure sensor, a flexible or rigid cable coupling the pressure sensor to interface electronics, a thin medical grade balloon encompassing the pressure sensor, and incompressible fluid provided in the balloon.

FIG. 2 is another schematic view of the sensor arrangement of FIG. 1.

FIGS. 3a-3e are schematic views of a process of putting the sensor arrangement of FIG. 1 together according to various steps depicted therein.

FIG. 4 is a photograph of an in vivo study of the sensor arrangement of FIG. 1.

FIG. 5 is a plot of normalized pressure from the pressure sensor arrangement of FIG. 1 vs. the normalized pressure from a gold standard.

FIG. 6 is a time-series of pressure baseline drift of the sensor arrangement of FIG. 1 compared to a reference sensor.

FIGS. 7a-7d are in-vivo pressure measurement results of two implanted packaged sensors according to the present disclosure (immediately post-surgical placement but prior to the start of longterm measurements), where FIGS. 7a and 7b show the pressure levels vs. time as compared to the readouts acquired by a reference system and FIGS. 7c and 7d show plots of pressure readings from the packaged sensors according to the present disclosure vs. reference pressure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

A novel low drift implantable pressure sensor arrangement is disclosed. The pressure sensor arrangement utilizes a packaging arrangement for implantable pressure sensors, which will significantly reduce drift (both base-line and sensitivity). The pressure sensor arrangement isolates the sensor from surrounding environments using a fluid filled balloon (or other elastomeric material). According to one embodiment, a pressure sensor is encapsulated in a medical grade thin balloon that is filled with silicone oil or other incompressible fluids. The medical grade balloon is sufficiently thin so as to transmit the external pressure to the pressure sensor without distortion while simultaneously isolates the sensor from body fluids.

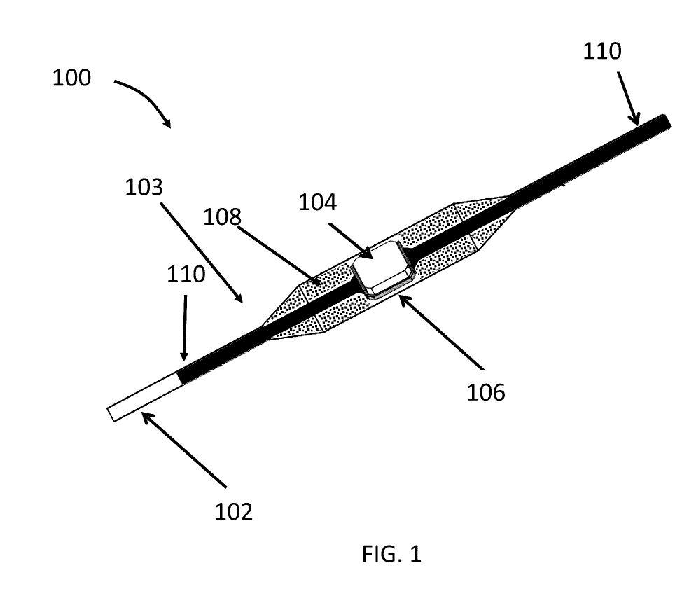

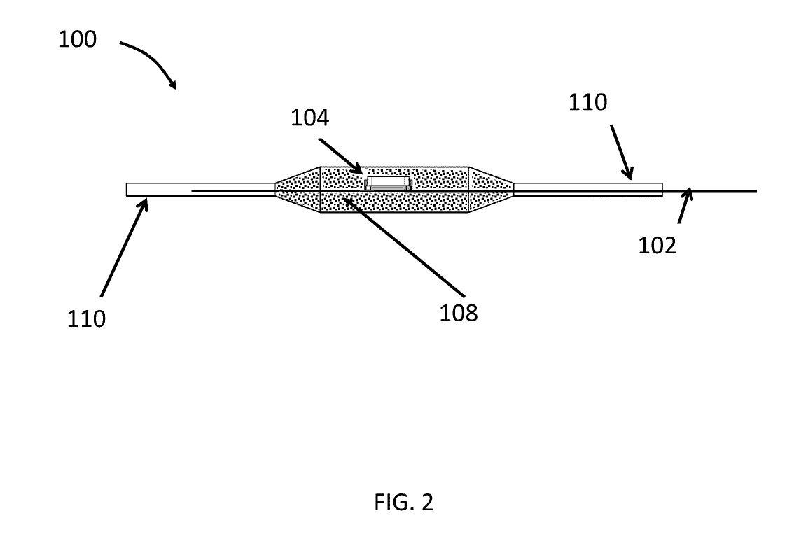

Referring to FIGS. 1 and 2 schematic views of a disclosed sensor arrangement 100 are provided. The sensor arrangement 100 includes: 1) a pressure sensor 104 (capacitive, piezoresistive, etc.), 2) a flexible or rigid cable 102 (also referred to as the sensor cable) coupling the pressure sensor 104 to an interface electronics provided in a package, e.g., ceramic, metal, or polymer (not shown), 3) a thin medical grade balloon 106, and 4) incompressible fluid 108. The pressure sensor 104 is coupled to the sensor cable 102 forming a sensor-cable subassembly 103 with the sensor cable 102 extending beyond the sensor 104 on at least one side of the sensor. The subassembly 103 is positioned inside the balloon 106 with the sensor cable 102 extending beyond the balloon 106 on at least one end of the balloon 106 for connectivity to the interface electronics (not shown). The balloon is filled with the incompressible fluid 108, e.g., silicone oil, encasing the portion of the sensor-cable subassembly 103 that is positioned inside the balloon. The balloon 106 is sized such that it can receive the sensor-cable subassembly 103 through an open end. Thereafter, once the subassembly 103 has been partially positioned inside the balloon with at least one distal end of the cable 102 positioned outside of the balloon 106, the open ends of the balloon are sealed providing sealed ends 110.

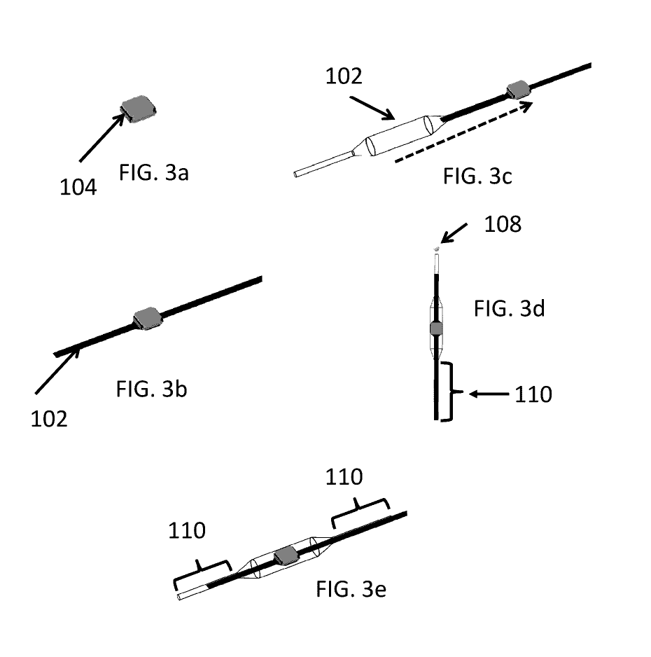

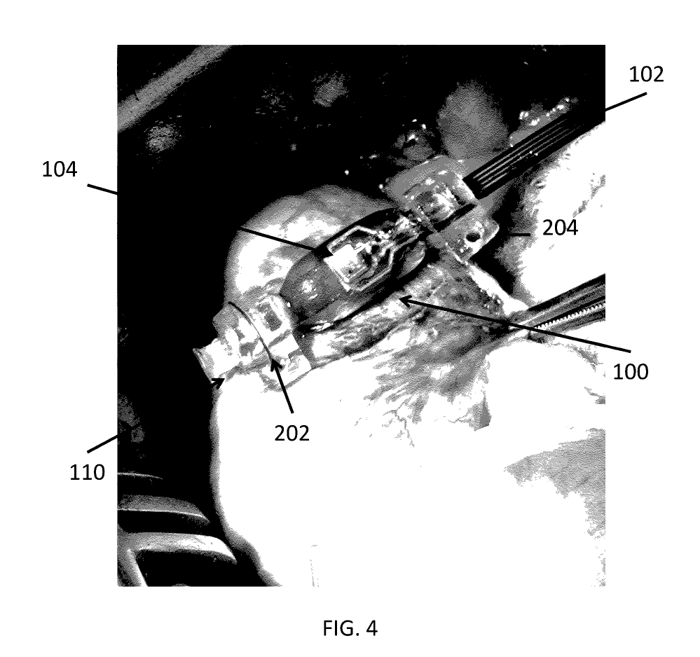

Referring to FIGS. 3a-3e a fabrication process of the sensor arrangement 100, according to the present disclosure is provided. The fabrication process starts by attaching the pressure sensor 104 to the interconnect cable (sensor cable) 102, with sufficient lengths of the cable 102 extending beyond the sensor for connectivity to an external circuit (not shown). The sensor 104 can be a piezoresistive or capacitive transducer, FIG. 3a. The sensor 104 is attached to the flexible or rigid interconnect cable (sensor cable) 102 using epoxy or other adhesives. It should be appreciated that the pressure sensor 104 may require two connections or more connections depending on the type of sensor that is used. For example, a capacitive sensor that utilizes changes in capacitance requires only two connections. Therefore, the sensor cable may only have two conductors or two traces. Alternatively, a piezoelectric sensor, may require fours connections, and therefore four traces or conductors. The connection of the sensor cable to the pressure sensor is shown in FIG. 3b. It should be appreciated that the cable 102 may extend from both sides of the pressure sensor 104 or only from one side for connectivity to external circuitry (not shown). If the cable 102 is extended from only one side, then the cable includes at least two conductors, one connected to one side of the sensor and the other connected to the other side of the sensor. If wire bonded, the bonds will be subsequently protected by applying a small amount of epoxy. The sensor 104 and interconnect cable 102 is then encapsulated by the thin medical grade balloon 106 and filled with the incompressible fluid 108, e.g., silicone oil. To do this, first the medical grade balloon 106, which has two open ends, is inserted from distal end of the flexible interconnect cable 102, FIG. 3c. The balloon passes over the sensor and cable, so it encapsulates the entire sensor and portions of the flexible cable 102. To ensure the silicone oil is entrapped in the balloon, proximal end of the balloon 106 (where it meets with the sensor cable 102) is first sealed against the interconnect cable using glue/adhesive or a pressure ring (not shown). Once the proximal end is sealed (i.e., sealed end 110), the balloon is filled with the incompressible fluid by injection from the distal open end of the balloon, FIG. 3d. Once sufficient amount of incompressible fluid is injected to fill the balloon up to the sealed proximal end 110, ensuring there are no air bubbles within the balloon 106, the distal end is sealed against the sensor cable using epoxy glue or other adhesives, completely sealing the sensor, FIG. 3e. As discussed above, while the figures thus far depict a balloon with two sealed ends where the sensor cable comes out of and is sealed at each end, the cable may come out of only one end (see FIG. 4 for a clearer view of this embodiment); or in an alternative embodiment, the balloon may only have one opening, where the sensor-cable subassembly 103 is inserted into the balloon from that opening.

Referring to FIG. 4, the sensor arrangement 100 according to the present disclosure is sutured to a pig's bladder wall using suture string 202 and implant anchor 204.

The disclosed low-drift sensor arrangement is experimentally validated for base-line and sensitivity drift. The sensing accuracy and drift was evaluated using in vitro experiments. The experiment was to simultaneously measure a pressure level using packaged sensor as well as a commercial pressure sensor as a reference standard and validate the correlation between the measurements. The packaged sensor was submerged in a container filled with saline solution (phosphate buffered solution) that mimics body fluid. The packaged pressure sensor was allowed to soak in saline for 70 days, while the pressure level was recorded every day. The pressure in the saline solution is affected by daily atmospheric pressure. Thus, the reference standard atmosphere pressure sensor was set next to the container and used to monitor the daily atmospheric pressure.

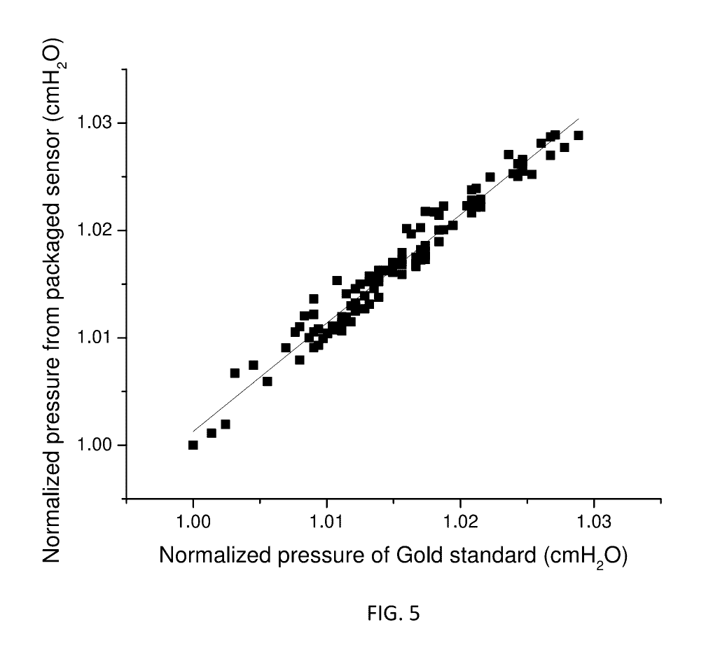

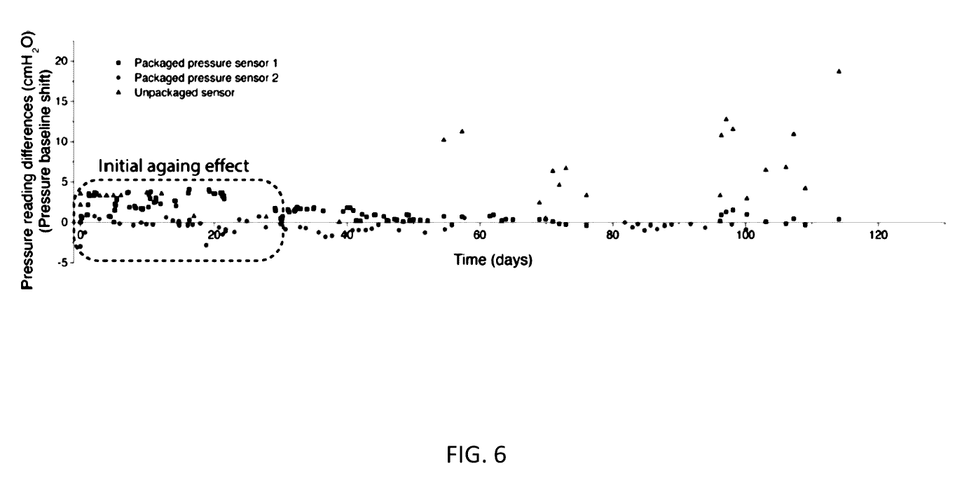

The normalized base-line pressure measurement of the disclosed low drift pressure sensor was plotted against a base-line pressure measurement of the reference standard pressure sensor, Referring to FIG. 5 (normalized pressure from the pressure sensor arrangement of the present disclosure vs. the normalized pressure from a gold standard) is a strong correlation between the two pressure sensors (i.e., the pressure sensor of the present disclosure and the “gold standard”). This graph plots show that the low drift implantable pressure sensor of the present disclosure maintains the base-line pressure over time compare to the reference standard. In vitro soak tests for 100 days using commercial micromachined piezoresistive pressure sensors demonstrate a stable operation with the output remaining within 1.8 cmH2O (1.3 mmHg) of a reference pressure transducer. FIG. 6 shows a time-series of pressure baseline drift compared to the reference sensor. Three packaged sensors according to the present disclosure were prepared and soaked in PBS solution. As can be seen, an initial aging effect with small fluctuations (±4 cmH2O) was observed in all three sensors in the first 30 days, settling down to a stable baseline after 40 days and remaining within 1.8 cmH2O (1.3 mmHg) of the reference.

Under similar test conditions, a non-isolated sensor fluctuates between 10 and 20 cmH2O (7.4-14.7 mmHg) of the reference, without ever settling to a stable operating regime. This indicates an on-going process, which is mostly due to the absorption of water into the packaging materials and resulting in slow degradation of certain materials under the ionic aqueous conditions. The source of the initial aging effect is partly due to the continuous operation of the sensor in the power-up mode which can result in certain aging process in the components, settling down after 30 days (<±2 cmH2O).

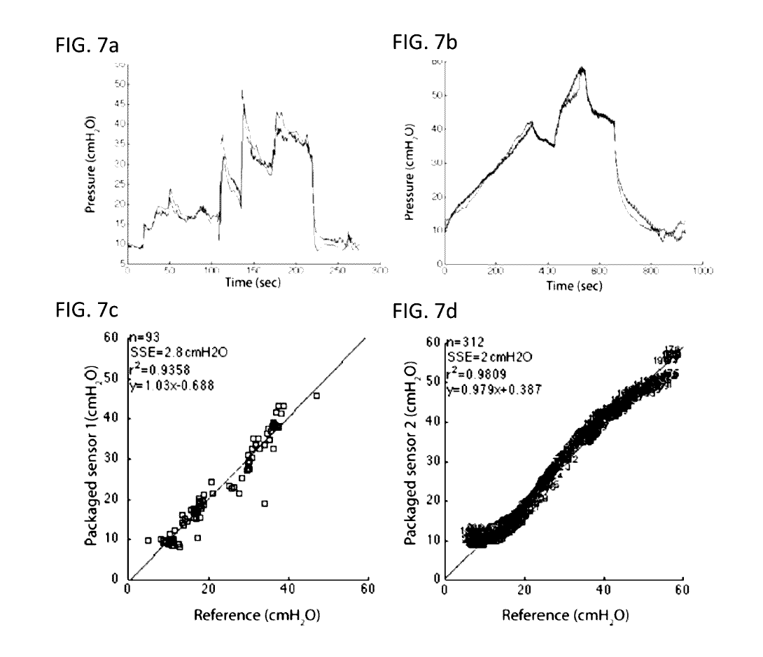

FIGS. 7a-67 show the in-vivo pressure measurement results of two implanted packaged sensors according to the present disclosure (immediately post-surgical placement but prior to the start of longterm measurements). FIGS. 7a and 7b show the pressure levels vs. time as compared to the readouts acquired by the urodynamic system (reference). The packaged sensors were able to closely track the intravesical measurements via the catheter-based sensor. The pressure measurements were also analyzed using the linear regression, FIGS. 7c and 7d (plots of pressure readings from the packaged sensors according to the present disclosure vs. reference pressure), with resulting slopes of 1.03 and 0.979 and R-square values of 0.93 and 0.98, respectively, indicating a strong correlation. After initial in-vivo calibrations and subsequent transfer of the animal to a care facility, the bladder pressures were wirelessly monitored for a period of 12 days.

While in the present disclosure an incompressible fluid is contemplated as the filling fluid for the balloon, it is within the scope of the present disclosure to use a compressible fluid governed by the Ideal Gas Law. In such an embodiment, drift and sensitivity of the sensor affected by changes in other parameters such as temperature must be taken into account in order to provide a sensor arrangement that provides the desired drift while provides sufficient sensitivity to changes in pressure.

Those skilled in the art will recognize that numerous modifications can be made to the specific implementations described above. The implementations should not be limited to the particular limitations described. Other implementations may be possible.

1. An implantable pressure sensor arrangement, comprising:
a pressure sensor;
a cable having a plurality of conductors coupled to the pressure sensor, forming a sensor-cable subassembly with the cable positioned about the sensor providing two ends;
a flexible balloon partially fitted around the sensor-cable subassembly, thereby encasing the pressure sensor while each end of the cable is extending away from the balloon;
the flexible balloon filled with an incompressible fluid, thereby allowing pressure changes outside of the balloon to be sensed by the pressure sensor inside of the balloon.

2. The implantable pressure sensor arrangement of claim 1, the pressure sensor is a capacitive pressure sensor operating based on changes in capacitance.

3. The implantable pressure sensor arrangement of claim 2, the cable includes a first conductor coupled to a first electrode of the pressure sensor and a second conductor coupled to a second electrode of the pressure sensor.

4. The implantable pressure sensor arrangement of claim 1, the incompressible fluid is silicone oil.
